(19) United States
(12) Patent Application Publication
(10) Pub. No.: US 2025/0191764 A1
(43) Pub. Date: Jun. 12, 2025

(54) DOMAIN-ORIENTED LLM COMPRESSION FOR MEDICAL DECISION MAKING

(71) Applicant: NEC Laboratories America, Inc., Princeton, NJ (US)

(72) Inventors: Yanchi Liu, Princeton, NJ (US); Wei Cheng, Princeton Junction, NJ (US); Xujiang Zhao, Hillsborough, NJ (US); Haifeng Chen, West Windsor, NJ (US); Zhengzhang Chen, Princeton Junction, NJ (US); Runxue Bao, Plainsboro, NJ (US)

(21) Appl. No.: 18/975,643

(22) Filed: Dec. 10, 2024

Related U.S. Application Data
(60) Provisional application No. 63/608,892, filed on Dec. 12, 2023.

(51) Int. Cl. G16H 50/20; G16H 10/60

(52) U.S. Cl. CPC G16H 50/20; G16H 10/60

(57) ABSTRACT

Methods and systems for model compression include determining importance values for respective parameters in a pre-trained model corresponding to general knowledge of the pre-trained model. Loss values are determined for removal of the parameters based on the importance values and a regularization term corresponding to domain-specific knowledge. Parameters are pruned from the pre-trained model based on the loss values to create a pruned model.

### RELATED APPLICATION INFORMATION

This application claims priority to U.S. Patent Application No. 63/608,892, filed on Dec. 12, 2023, incorporated herein by reference in its entirety.

### BACKGROUND

### Technical Field

The present invention relates to machine learning models and, more particularly, to domain compression of large language models.

### Description of the Related Art

Pre-trained large language models (LLMs) are well suited to a variety of natural language processing tasks. However, such models are pre-trained on an open-domain corpus and are fine-tuned for generic tasks. They therefore have less accuracy when performing domain-dependent tasks, where the vocabulary and grammar may be different from what was available during their training. Fine-tuning and compressing such models in the context of a particular domain can be very computationally expensive.

### SUMMARY

A method for model compression includes determining importance values for respective parameters in a pre-trained model corresponding to general knowledge of the pre-trained model. Loss values are determined for removal of the parameters based on the importance values and a regularization term corresponding to domain-specific knowledge. Parameters are pruned from the pre-trained model based on the loss values to create a pruned model.

A system for model compression includes a hardware processor and a memory that stores a computer program. When executed by the hardware processor, the computer program causes the hardware processor to determine importance values for respective parameters in a pre-trained model corresponding to general knowledge of the pre-trained model, to determine loss values for removal of the parameters based on the importance values and a regularization term corresponding to domain-specific knowledge, and to prune parameters from the pre-trained model based on the loss values to create a pruned model.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a diagram illustrating a model compression process that includes pruning of parameters, in accordance with an embodiment of the present invention;

FIG. 2 is a block/flow diagram of a method for model compression, in accordance with an embodiment of the present invention;

FIG. 3 is a block/flow diagram of a method for compressing and using a model to perform a task, in accordance with an embodiment of the present invention;

FIG. 4 is a block diagram of a healthcare facility that makes use of a compressed large language model to perform patient diagnosis and treatment, in accordance with an embodiment of the present invention;

FIG. 5 is a block diagram of a computing device that can compress a model and correct a patient's treatment, in accordance with an embodiment of the present invention;

FIG. 6 is a diagram of an exemplary neural network architecture that can be used to implement a large language model, in accordance with an embodiment of the present invention; and

FIG. 7 is a diagram of an exemplary deep neural network architecture that can be used to implement a large language model, in accordance with an embodiment of the present invention.

### DETAILED DESCRIPTION OF PREFERRED EMBODIMENTS

A large language model (LLM) can be compressed in a cost-efficient, domain-oriented manner by pruning unnecessary weights for general knowledge and domain knowledge, and then fine-tuning the pruned model in a parameter-efficient way. Both the pruning and fine-tuning may be performed in an efficient manner. In this way, a compressed LLM with many fewer parameters can be generated for a targeted domain.

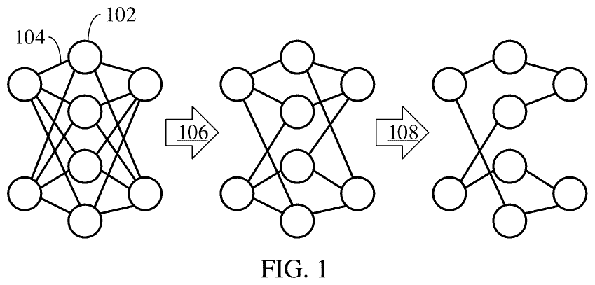

Referring now to FIG. 1, a diagram of domain-oriented model compression is shown. An exemplary neural network model is shown with a set of neurons 102 that are connected by weights 104. As will be described in greater detail below, each neuron 102 implemented a predetermined function on its inputs and generates a corresponding output. The weights 104 are applied to the neuron outputs, for example by multiplication, to create the input to the next layer of neurons 102. Outputs from multiple neurons in a given layer may be combined with a weighted summation to make an input to the next layer.

The weights 104 correspond to the parameters of the model. During training, the values of these weights 104 are adjusted to improve the model's accuracy in performing a given task. A pre-trained LLM may have trillions of such parameters. The computational cost of performing a task with the model rises with the number of parameters, for example as each parameter may represent a separate computation to perform.

To decrease the number of parameters, a pruning process 106 may be performed. This pruning 106 identifies parameters which are not useful in the performance of a particular domain-specific task. The identified parameters may be removed from the model, so that the corresponding computation may be skipped when the model is used. A fine-tuning process 108 can then further compress the model by fine-tuning it for the domain-specific task.

Two types of knowledge may be encoded in the parameters of a pre-trained LLM, which need to be preserved in the compressed model. Domain-shared knowledge represents general knowledge that enables the LLM to process natural language and express itself in a humanlike manner. Domain-specific knowledge enables the LLM to respond like a domain expert. A dual pruning mechanism can capture the weights that are important to these two types of knowledge and can prune unimportant weights.

General knowledge may be located within the model by evaluating the importance of model weights to general knowledge, while domain knowledge can be located by evaluating the importance of model weights to domain knowledge. Importance may be determined using training weight gradients. These importances may be integrated to evaluate a given weight's overall importance.

In some cases, the general knowledge may be derived from a large and undifferentiated corpus, for example drawing from any publicly available texts. In some cases the general knowledge may be derived from a more limited domain, such as medical information from journal and online encyclopedia sources. The domain-specific knowledge may be derived from a particular medical specialty, such as endocrinology or cardiology, and may further include domain-specific sources such as clinical notes and diagnostic reports specific to particular medical specialties.

Once the model has been pruned, a domain dataset is used to fine-tune 108 the model in a parameter-efficient way. In this way a compressed domain-specific model is generated that preserves the multi-task-solving capability of the model in the target domain.

An example of a domain-specific task is the processing of medical records in a healthcare context. For example, an LLM may be used to perform question-answering for diagnosis, where a patient's symptoms and test results are provided as context. The LLM may be fine-tuned with domain-specific knowledge that relates to the medical context and so can answer questions that a generic pre-trained LLM would not be able to handle.

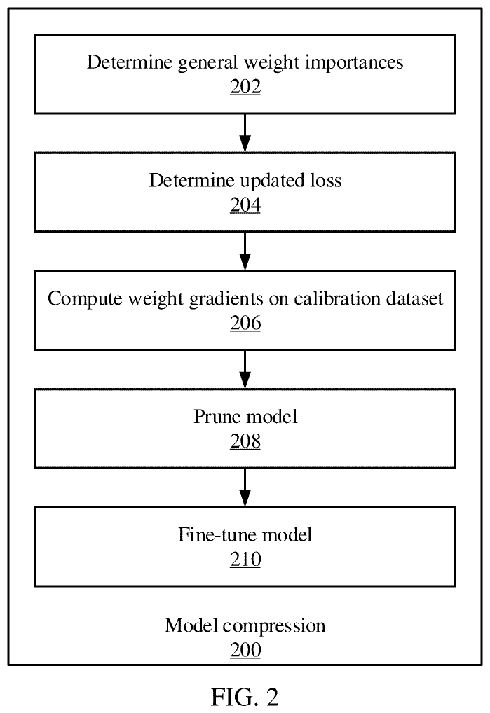

Referring now to FIG. 2, a method of compressing 200 a pre-trained LLM is shown. Block 202 determines general weight importances for domain-shared knowledge, which maintains the linguistic capabilities and multi-task solving capabilities of the LLM. If the model were to lose its basic linguistic capabilities during the compression process, it may not be able to generate coherent responses.

Block 202 operates on the assumption that important weights will cause a relatively large increase in loss value when pruned than those which are less important. If a calibration dataset of general domains Dg={xⱼ, yⱼ}ⱼ₌₁^{N}, with N being a size used for training, x being an input for a training example j and y, being an associated output, the importance of each weight at index m can be approximated using a Taylor series:

$${I}_{{W}^{m}}^{m} = \vert ℒ ( {𝒟}_{g} ) - {ℒ}_{{W}^{m} = 0} ( {𝒟}_{g} ) \vert = \vert \frac{\partial ℒ ( {𝒟}_{g} )}{\partial {W}^{m}} {W}^{m} + \frac{1}{2} {W}^{m} {H}_{m m} {W}^{m} + O ( {‖ {W}^{m} ‖}^{3} ) \vert$$

where H denotes the Hessian matrix and [symbol] is the cross-entropy loss. For a model that is trained to a local minimum on its loss curvature, such as a pre-trained LLM, the importance of W^{m} may be approximated as:

$${ε}^{m} = \frac{1}{2} \frac{{( {W}^{m} )}^{2}}{{[ {H}^{- 1} ]}_{m m}}$$

The importance ε^{m} may be interpreted as the error caused by removing the weight W^{m} The importance ε^{m} may be computed for all weights subject to pruning to construct a matrix of importance scores G with respect to the general domains, having the same dimensions as W.

Block 204 determines an updated loss function that incorporates the general weight importances. The loss function for LLM training may be modified to identify the weights that are important for both the general and domain-specific knowledge. The cross-entropy loss may be used when training a next token prediction task. A regularization term may be added to constrain the change of important general weights found in block 202. If there are M prunable weights, to train on a domain-specific calibration dataset Dₛ, then the regularization term is added on top of the next token prediction loss [symbol] to obtain a final training objective:

$${ℒ}_{n e w} = {ℒ}_{next} + λ {\sum }_{m = 1}^{M} {{G}^{m} ( {W}^{{m}^{′}} - {W}^{m} )}^{2}$$

where G^{m} is the general weight importance, W^{m}′ denotes the updated weight value of W^{m}, λ is a weighting hyperparameter, and the second term on the right is [symbol] .

In practice, the direct calculation of this regularization term in the forward pass is computationally expensive because it involves both W^{m} and G^{m}, which can be very large, and because gathering the updated model parameters in a partitioned or sharded system is inefficient. Gradient descent maybe used to optimize these parameters. At a learning rate a, with the gradient of each parameter with respect to [symbol] being denoted as gₙₑₓₜ^{m}, the regularization term may be reduced to:

$$\begin{matrix}{ℒ}_{regular} = {\sum }_{m = 1}^{M} {{G}^{m} ( {W}^{m ′} - {W}^{m} )}^{2} \\ = {\sum }_{m = 1}^{M} {{G}^{m} ( {W}^{m} - α {g}_{n e x t}^{m} - {W}^{m} )}^{2} \\ = {\sum }_{m = 1}^{M} {α}^{2} {{G}^{m} ( {g}_{n e x t}^{m} )}^{2}\end{matrix}$$

where α is a learning rate.

During the backward pass, optimizing this regularization term uses second-order derivatives, which indicates that Hessian matrices (H) are needed. Directly computing the Hessian matrices is infeasible for such a large number of parameters. Therefore, the Fisher information matrix may be used to approximate the diagonal of the Hessian. The Fisher information matrix can be further approximated by the average of the squared gradient of the model's prediction over P. The gradient of the regularization is determined with respect to every parameter matrix in a finer granularity:

$$\begin{matrix}\frac{\partial {ℒ}_{regular}}{\partial {W}^{m}} \approx 2 λ {α}^{2} {G}^{m} {g}_{n e x t}^{m} {H}_{m m} \\ {H}_{m m} \approx \frac{1}{P} {\sum }_{j = 1}^{P} {( {g}_{next}^{m} ( {x}_{j} , {y}_{j} ) )}^{2}\end{matrix}$$

The derivative

$$\frac{\partial {ℒ}_{regular}}{\partial {W}^{m}}$$

may be determined instead of relying on a backward pass to maximize computational efficiency. The final gradient computation of the regularized loss function may be expressed as:

$$\frac{\partial {ℒ}_{n e w}}{\partial {W}^{m}} = \frac{\partial {ℒ}_{n e x t}}{\partial {W}^{m}} + \frac{\partial {ℒ}_{regular}}{\partial {W}^{m}}$$

Block 206 computes weight gradients on a calibration dataset to generate a dual-pruning importance score for each weight. The importance may be calculated as I_{W}^{m}, as described above, rather than as ε^{m}, because the model has not yet converged to an optimum on the target domain. However, direct computation of the Hessian matrix is infeasible as it has O(M²) complexity for each weight update. Therefore the diagonal of the Hessian may be approximated to generate a final importance score:

$${S}^{m} \approx \vert \frac{\partial {ℒ}_{n e w} ( {𝒟}_{s} )}{\partial {W}^{m}} {W}^{m} + {\frac{1}{2} [ \frac{\partial {ℒ}_{n e w} ( {𝒟}_{s} )}{\partial {W}^{m}} {W}^{m} ]}^{2} + O ( {‖ {W}^{m} ‖}^{3} ) \vert$$

where [symbol] is a domain-specific calibration dataset. The term O(∥W^{m}∥³) can be neglected according to the quadratic approximation. The calculation of S™ considers both general and domain-specific knowledge via the regularized training objective. Combining both regularization and importance estimation via the empirical Fisher approximation, pruning 208 maintains weights important to both general and domain-specific knowledge, thereby preserving generality and specificity. For example, a target sparsity for the pruning may be 50%, in which case the 50% of the weights of each layer in the model having the lowest importance scores may be pruned, for example by setting those parameters to zero.

After pruning is performed, the remaining parameters of the model are fine-tuned in block 210 for a domain-specific task. Examples of tasks that the pruned model may be fine-tuned for include information extraction relating to a particular medical specialty (e.g., symptoms and treatment methods) and question answering.

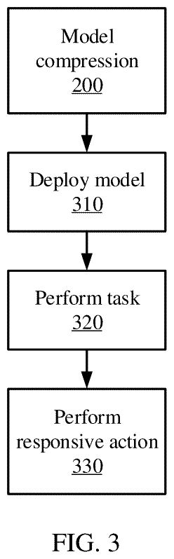

Referring now to FIG. 3, a method for compressing and using a machine learning model is shown. Block 200 performs model compression on a pre-trained LLM, for example using a domain-specific dataset to identify parameters of the pre-trained LLM that are important for the target domain and those which are important to general knowledge of the LLM and to prune parameters which are less important. The compression 200 may further fine-tune the pruned model for the target domain and/or a particular task therein.

Block 310 then deploys the compressed model. In some cases, where the task is to be performed at the same system where the compression is performed, the deployment 310 may be omitted. Deploying the model may include copying the remaining parameters of the compressed model to a target system where the task is to be performed. Because the compressed model is substantially smaller than the original pre-trained model, the target system may have fewer resources (e.g., processor power, storage space, and system memory) than would be needed to execute the pre-trained model.

Block 320 then performs the task on newly acquired data. For example, in the context of a healthcare task, prompts may be applied to the compressed model which provide recent test results for a patient to elicit information about a potential diagnosis and treatment. Again, because the compressed model is smaller than the pre-trained model, it can be executed faster than the pre-trained model and can furthermore be used in systems that would be incapable of running the pre-trained model, such as embedded systems with limited system memory.

Block 330 then performs an action that is responsive to the output of the compressed model. For example, if the model indicates a particular diagnosis or treatment, the responsive action may include automatically administering the treatment.

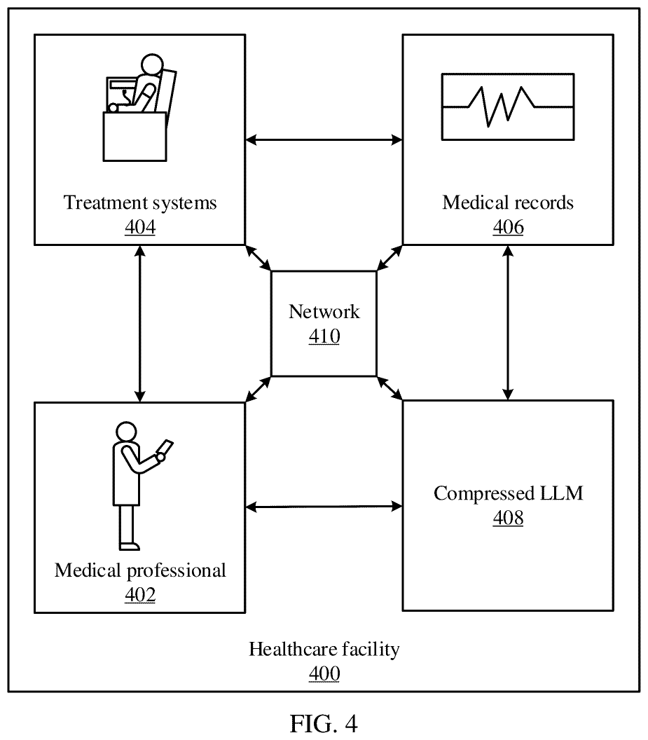

Referring now to FIG. 4, a diagram of information extraction is shown in the context of a healthcare facility 400. A compressed LLM 408 may be used to process information about a patient's medical history to aid with medical decision making and diagnosis. The compressed LLM 408 may be used to answer questions relating to a patient's medical condition based on up-to-date medical records 406.

The healthcare facility may include one or more medical professionals 402 who review information extracted from a patient's medical records 406 to determine their healthcare and treatment needs. These medical records 406 may include self-reported information from the patient, test results, and notes by healthcare personnel made to the patient's file. Treatment systems 404 may furthermore monitor patient status to generate medical records 406 and may be designed to automatically administer and adjust treatments as needed.

Based on information provided by the compressed LLM 408, the medical professionals 402 may make medical decisions about patient healthcare suited to the patient's needs. For example, the medical professionals 402 may make a diagnosis of the patient's health condition and may prescribe particular medications, surgeries, and/or therapies.

The different elements of the healthcare facility 400 may communicate with one another via a network 410, for example using any appropriate wired or wireless communications protocol and medium. Thus the compressed LLM 408 can receive a query from medical professionals 402 relating to a condition and may formulate a response based on information gleaned from stored medical records 406. The compressed LLM 408 may coordinate with treatment systems 404 in some cases to automatically administer or alter a treatment. For example, if the compressed LLM 408 indicates a particular disease or condition, then the treatment systems 404 may automatically halt the administration of the treatment.

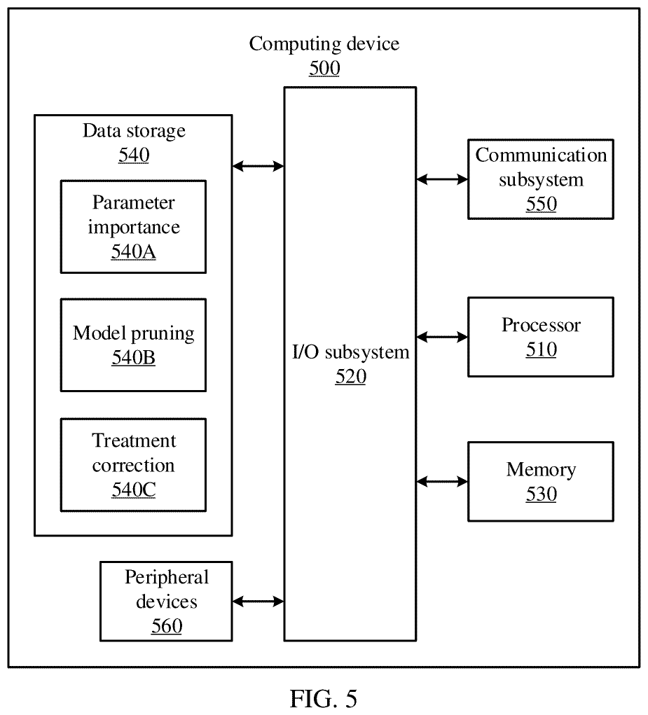

As shown in FIG. 5, the computing device 500 illustratively includes the processor 510, an input/output subsystem 520, a memory 530, a data storage device 540, and a communication subsystem 550, and/or other components and devices commonly found in a server or similar computing device. The computing device 500 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 530, or portions thereof, may be incorporated in the processor 510 in some embodiments.

The processor 510 may be embodied as any type of processor capable of performing the functions described herein. The processor 510 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 530 may be embodied as any type of volatile or non-volatile memory or data storage capable of performing the functions described herein. In operation, the memory 530 may store various data and software used during operation of the computing device 500, such as operating systems, applications, programs, libraries, and drivers. The memory 530 is communicatively coupled to the processor 510 via the I/O subsystem 520, which may be embodied as circuitry and/or components to facilitate input/output operations with the processor 510, the memory 530, and other components of the computing device 500. For example, the I/O subsystem 520 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 520 may form a portion of a system-on-a-chip (SOC) and be incorporated, along with the processor 510, the memory 530, and other components of the computing device 500, on a single integrated circuit chip.

The data storage device 540 may be embodied as any type of device or devices configured for short-term or long-term storage of data such as, for example, memory devices and circuits, memory cards, hard disk drives, solid state drives, or other data storage devices. The data storage device 540 can store program code 540A for determining parameter importance, 540B for pruning a pre-trained model according to parameter importance, and/or 540C for correcting a patient's treatment based on inputs to the model. Any or all of these program code blocks may be included in a given computing system. The communication subsystem 550 of the computing device 500 may be embodied as any network interface controller or other communication circuit, device, or collection thereof, capable of enabling communications between the computing device 500 and other remote devices over a network. The communication subsystem 550 may be configured to use any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 500 may also include one or more peripheral devices 560. The peripheral devices 560 may include any number of additional input/output devices, interface devices, and/or other peripheral devices. For example, in some embodiments, the peripheral devices 560 may include a display, touch screen, graphics circuitry, keyboard, mouse, speaker system, microphone, network interface, and/or other input/output devices, interface devices, and/or peripheral devices.

Of course, the computing device 500 may also include other elements (not shown), as readily contemplated by one of skill in the art, as well as omit certain elements. For example, various other sensors, input devices, and/or output devices can be included in computing device 500, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized. These and other variations of the processing system 500 are readily contemplated by one of ordinary skill in the art given the teachings of the present invention provided herein.

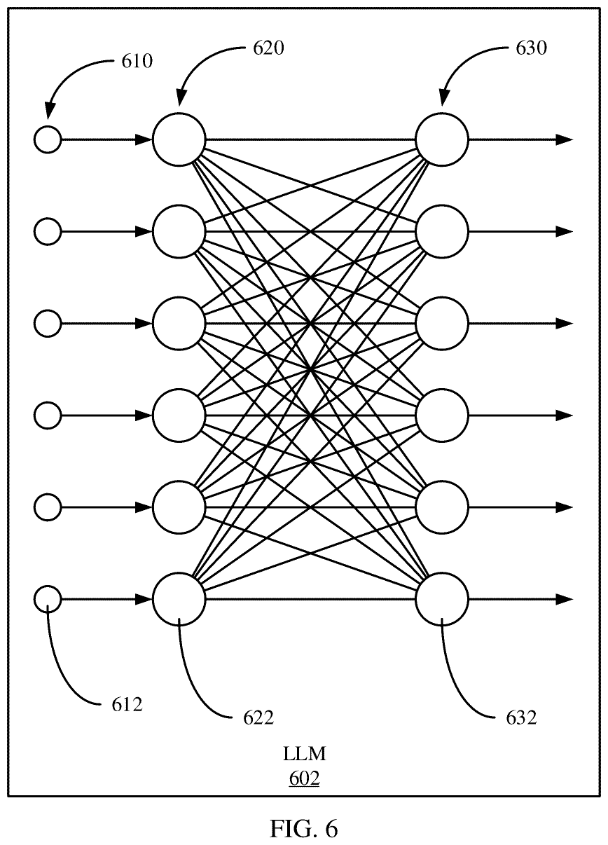

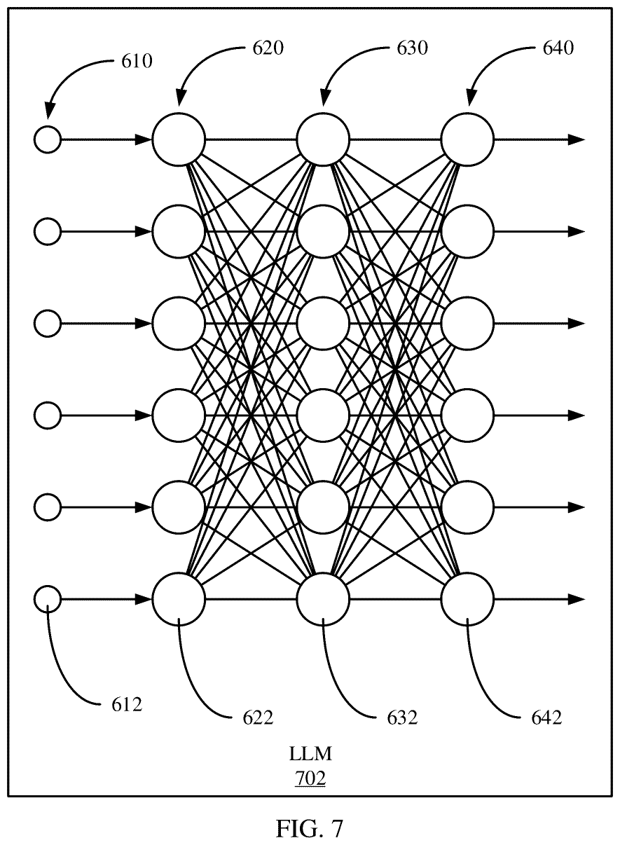

Referring now to FIGS. 6 and 7, exemplary neural network architectures are shown, which may be used to implement parts of the present models, such as the LLM 602/702. A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the input data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types, and may include multiple distinct values. The network can have one input node for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples, and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

In layered neural networks, nodes are arranged in the form of layers. An exemplary simple neural network has an input layer 620 of source nodes 622, and a single computation layer 630 having one or more computation nodes 632 that also act as output nodes, where there is a single computation node 632 for each possible category into which the input example could be classified. An input layer 620 can have a number of source nodes 622 equal to the number of data values 612 in the input data 610. The data values 612 in the input data 610 can be represented as a column vector. Each computation node 632 in the computation layer 630 generates a linear combination of weighted values from the input data 610 fed into input nodes 620, and applies a non-linear activation function that is differentiable to the sum. The exemplary simple neural network can perform classification on linearly separable examples (e.g., patterns).

A deep neural network, such as a multilayer perceptron, can have an input layer 620 of source nodes 622, one or more computation layer(s) 630 having one or more computation nodes 632, and an output layer 640, where there is a single output node 642 for each possible category into which the input example could be classified. An input layer 620 can have a number of source nodes 622 equal to the number of data values 612 in the input data 610. The computation nodes 632 in the computation layer(s) 630 can also be referred to as hidden layers, because they are between the source nodes 622 and output node(s) 642 and are not directly observed. Each node 632, 642 in a computation layer generates a linear combination of weighted values from the values output from the nodes in a previous layer, and applies a non-linear activation function that is differentiable over the range of the linear combination. The weights applied to the value from each previous node can be denoted, for example, by w₁, w₂, . . . wₙ₋₁, wₙ. The output layer provides the overall response of the network to the input data. A deep neural network can be fully connected, where each node in a computational layer is connected to all other nodes in the previous layer, or may have other configurations of connections between layers. If links between nodes are missing, the network is referred to as partially connected.

Training a deep neural network can involve two phases, a forward phase where the weights of each node are fixed and the input propagates through the network, and a backwards phase where an error value is propagated backwards through the network and weight values are updated.

The computation nodes 632 in the one or more computation (hidden) layer(s) 630 perform a nonlinear transformation on the input data 612 that generates a feature space. The classes or categories may be more easily separated in the feature space than in the original data space.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method for model compression, comprising:
determining importance values for respective parameters in a pre-trained model corresponding to general knowledge of the pre-trained model;
determining loss values for removal of the parameters based on the importance values and a regularization term corresponding to domain-specific knowledge; and
pruning parameters from the pre-trained model based on the loss values to create a pruned model.

2. The method of claim 1, wherein pruning includes setting a predetermined percentage of the parameters to zero based on their relative loss values.

3. The method of claim 1, wherein determining the loss values includes determining a gradient of the regularization term with respect to the parameters.

4. The method of claim 1, wherein the pre-trained model is a large language model implemented as a machine learning system.

5. The method of claim 1, further comprising fine-tuning the pruned model for a target domain.

6. The method of claim 1, wherein the loss values are determined as:
$${S}^{m} \approx \vert \frac{\partial {ℒ}_{n e w} ( {𝒟}_{s} )}{\partial {W}^{m}} {W}^{m} + {\frac{1}{2} [ \frac{\partial {ℒ}_{n e w} ( {𝒟}_{s} )}{\partial {W}^{m}} {W}^{m} ]}^{2} \vert$$
where [symbol] is a loss function that combines a next token prediction loss with the regularization term, W^{m} are the parameters, and Dₛ is a domain-specific dataset.

7. The method of claim 1, further comprising performing a medical diagnosis task using the pruned model.

8. The method of claim 7, wherein the medical diagnosis task includes determining a healthcare condition for a patient based on input information about the patient.

9. The method of claim 8, further comprising automatically performing a treatment action for the patient responsive to the medical diagnosis.

10. The method of claim 1, further comprising executing the pruned model using inputs in a target domain.

11. A system for model compression, comprising:
a hardware processor; and
a memory that stores a computer program which, when executed by the hardware processor, causes the hardware processor to:
determine importance values for respective parameters in a pre-trained model corresponding to general knowledge of the pre-trained model;
determine loss values for removal of the parameters based on the importance values and a regularization term corresponding to domain-specific knowledge; and
prune parameters from the pre-trained model based on the loss values to create a pruned model.

12. The system of claim 11, wherein the pruning includes setting a predetermined percentage of the parameters to zero based on their relative loss values.

13. The system of claim 11, wherein the determination of the loss values includes determination a gradient of the regularization term with respect to the parameters.

14. The system of claim 11, wherein the pre-trained model is a large language model implemented as a machine learning system.

15. The system of claim 11, wherein the computer program further causes the hardware processor to fine-tune the pruned model for a target domain.

16. The system of claim 11, wherein the loss values are determined as:
$${S}^{m} \approx \vert \frac{\partial {ℒ}_{n e w} ( {𝒟}_{s} )}{\partial {W}^{m}} {W}^{m} + {\frac{1}{2} [ \frac{\partial {ℒ}_{n e w} ( {𝒟}_{s} )}{\partial {W}^{m}} {W}^{m} ]}^{2} \vert$$
where [symbol] is a loss function that combines a next token prediction loss with the regularization term, W^{m} are the parameters, and Dₛ is a domain-specific dataset.

17. The system of claim 11, wherein the computer program further causes the hardware processor to perform a medical diagnosis task using the pruned model.

18. The system of claim 17, wherein the medical diagnosis task includes determining a healthcare condition for a patient based on input information about the patient.

19. The system of claim 18, wherein the computer program further causes the hardware processor to automatically perform a treatment action for the patient responsive to the medical diagnosis.

20. The system of claim 11, wherein the computer program further causes the hardware processor to execute the pruned model using inputs in a target domain.
